(19) United States
(12) Patent Application Publication
(10) Pub. No.: US 2022/0015714 A1
(43) Pub. Date: Jan. 20, 2022

(54) Methods For Determining Disease Risk Combining Downsampling of Class-Imbalanced Sets With Survival Analysis

(71) Applicant: SOMALOGIC, INC., Boulder, CO (US)

(72) Inventors: Yolanda Hagar, Boulder, CO (US); Gargi Datta, Boulder, CO (US); Leigh E. Alexander, Boulder, CO (US); Michael A. Hinterberg, Boulder, CO (US)

(73) Assignee: SOMALOGIC, INC., Boulder, CO (US)

(21) Appl. No.: 17/297,669

(22) PCT Filed: Nov. 21, 2019
(86) PCT No.: PCT/US2019/062561

§ 371 Date: May 27, 2021

Related U.S. Application Data
(60) Provisional application No. 62/773,028, filed on Nov. 29, 2018.
(60) Provisional application No. 62/783,733, filed on Dec. 21, 2018.

(51) Int. Cl. A61B 5/00; G16H 50/30; G16H 50/20; A61B 5/145; A61B 5/021

(52) U.S. Cl. CPC A61B 5/7275; G16H 50/30; G16H 50/20; A61B 5/4866; A61B 5/14546; A61B 5/021; A61B 5/4872; A61B 5/7221

(57) ABSTRACT

A method for downsampling class-imbalanced sets with survival analysis comprising: acquiring a class-imbalanced data set, wherein the class-imbalanced data set comprises biological data from a plurality of subjects, wherein the biological data of each subject includes an observation, a time value, and a plurality of clinical measurements, and wherein the biological data is categorized as being part of a majority data class or a minority data class, wherein the majority data class has a greater number of observations than the minority data class; downsampling the class-imbalanced data set, wherein the downsampling results in the majority data class having an equivalent or substantially equivalent number of observations as the minority data class; and performing cross-validation on the downsampled data set with a survival analysis to generate a survival model, wherein the observation comprises an event or no event at a specific time value.

### CROSS-REFERENCE TO RELATED APPLICATIONS

This application claims the benefit of priority to U.S. Provisional Patent Application No. 62/773,028, filed Nov. 29, 2018, and U.S. Provisional Patent Application No. 62/783,733, filed Dec. 21, 2018, which are incorporated by reference herein, in their entirety.

### TECHNICAL FIELD

The present disclosure relates generally to the field of disease risk determination and, more particularly, to systems and methods for processing electronic data to determine disease risk.

### BACKGROUND

Methods for identifying biomarkers associated with the risk of various disease related conditions or events, e.g. cardiovascular events, diabetes diagnoses, various cancer types, etc. have improved primarily due to the discovery of high throughput technologies such as gene sequencing, transcriptomics, proteomics and metabolomics. However, these technologies also complicate matters by providing high-dimensional data that represents complex biological processes that can make it difficult to extract meaningful biomarker signatures.

When the primary goal is the correct identification of individuals who will experience a disease related condition or event within a specified period of time, an analysis that typically would only employ classification approaches can be strengthened by framing it as a special type of classification problem that incorporates both survival model approaches in conjunction with classification tools. However, survival analysis can suffer from an imbalance between the number of patients who do and do not experience the disease related condition or event. It is known that predictive classifiers generally perform poorly on imbalanced data, as the model is trained to be accurate “as often as possible.” This effect occurs because the larger, majority class drives the features selected for the model, as the minority class can be mis-classified frequently while the majority class is still predicted accurately. However, the sensitivity and specificity will become imbalanced, such that one is maximized over the other depending on which group has a greater number of observations. In modeling health outcomes, it is common to have low disease prevalence within a cohort, forming the minority class. In that situation, specificity will be maximized at the expense of sensitivity, which is problematic when the goal is to identify as many individuals as possible who are at risk for development of a condition or an event.

Therefore, there continues to be a need for alternative methods for improved ways to identify molecular signatures or biomarkers for a particular disease or condition. The present disclosure meets such needs by providing methods for improving biomarker discovery.

### SUMMARY

According to some aspects of the present disclosure, systems and methods disclosed relate to downsampling a majority class, i.e. the class with more observations, of a class-imbalanced data set comprising a time value in order to improve the sensitivity and specificity in survival analysis. The aim of downsampling is to “bias” the classifier so that it pays equal attention to the diagnosed and non-diagnosed individuals to balance the sensitivity and specificity of the model.

In one embodiment, a method is disclosed comprising: acquiring a class-imbalanced data set, wherein the class-imbalanced data set comprises biological data from a plurality of subjects, wherein the biological data of each subject includes an observation, a time value and a plurality of clinical measurements, and wherein the biological data is categorized as being part of a majority data class or a minority data class, wherein the majority data class has a greater number of observations than the minority data class; downsampling the class-imbalanced data set to generate a downsampled data set, wherein the downsampling results in the majority data class having an equivalent or substantially equivalent number of observations as the minority data class; and performing cross-validation on the downsampled data set with a survival analysis to generate a survival model; wherein, the observation comprises an event or no event at a specific time value.

According to aspects of the present disclosure, an area under the curve (AUC), sensitivity, specificity, and/or C-index of the survival model is closer to 1 than an AUC, sensitivity, specificity, and/or C-index of a survival model where the class-imbalanced data set was not downsampled prior to the survival analysis.

In other examples, the class-imbalanced data set is a survival data set and/or the event is a disease, disorder, or condition of a subject. In further examples, the survival analysis is selected from the group consisting of a cox proportional hazard analysis, a random forest analysis, accelerated failure time analysis, and any combination thereof, including machine learning adaptations such as penalized regression techniques. The method may further comprise an elastic net penalty.

In other embodiments, the cross-validation is at least a 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold cross-validation. In other embodiments, the survival model comprises from 5 to 1,000 features, wherein each feature is selected from the group consisting of a protein measurement, a clinical factor, and a combination thereof. The clinical factor is selected from the group consisting of age, weight, blood pressure, height, BMI, cholesterol, sex, and a combination thereof.

In further embodiments, the clinical measurements are selected from proteomic measurements, genomic measurements, transcriptome measurements, metabolomics measurements, and a combination thereof. Further, the cross-validation is selected form k-fold, Generalized Monte Carlo, leave-p-out cross-validation, or bootstrapping methods.

According to aspects of the present disclosure, the majority data class is 95% of the class-imbalanced data set and the minority data class is 5% of the class-imbalance data set, or the majority data class is 90% of the class-imbalanced data set and the minority data class is 10% of the class-imbalance data set, or the majority data class is 85% of the class-imbalanced data set and the minority data class is 15% of the class-imbalance data set, or the majority data class is 80% of the class-imbalanced data set and the minority data class is 20% of the class-imbalance data set, or the majority data class is 75% of the class-imbalanced data set and the minority data class is 25% of the class-imbalance data set, or the majority data class is 70% of the class-imbalanced data set and the minority data class is 30% of the class-imbalance data set, or the majority data class is 65% of the class-imbalanced data set and the minority data class is 35% of the class-imbalance data set, or the majority data class is 60% of the class-imbalanced data set and the minority data class is 40% of the class-imbalance data set.

In accordance with another embodiment, a method is disclosed comprising: downsampling a class-imbalanced data set to generate a downsampled data set, wherein the downsampling results in a majority data class having an equivalent or substantially equivalent number of observations as a minority data class; and performing cross-validation on the downsampled data set with a survival analysis to generate a survival model; wherein, the observation comprises an event or no event at a specific time value; wherein, the class-imbalanced data set comprises biological data from a plurality of subjects, wherein the biological data of each subject includes an observation, a time value, and a plurality of protein measurements, and wherein the biological data is categorized as being part of the majority data class or the minority data class, wherein the majority data class has a greater number of observations than the minority data class.

According to aspects of the present disclosure, an AUC, sensitivity, specificity, and/or C-index of the survival model is closer to 1 than an AUC, sensitivity, specificity, and/or C-index of a survival model where the class-imbalanced data set was not downsampled prior to the survival analysis.

In examples of the disclosure, the AUC is calculated based on the determination of whether or not a subject will have an event by a specified time-point.

A computer-implemented method for determining disease risk is also disclosed, the method comprising: acquiring a class-imbalanced data set, wherein the class-imbalanced data set comprises biological data from a plurality of subjects, wherein the biological data of each subject includes an observation, a time value, and a plurality of clinical measurements, and wherein the biological data is categorized as being part of a majority data class or a minority data class, wherein the majority data class has a greater number of observations than the minority data class; downsampling the class-imbalanced data set to generate a downsampled data set, wherein the downsampling results in the majority data class having an equivalent or substantially equivalent number of observations as the minority data class; and performing cross-validation on the downsampled data set with a survival analysis to generate a survival model; wherein, the observation comprises an event or no event at a specific time value; and the steps of downsampling and cross-validation are computed with a computer system.

According to aspects of the present disclosure, an AUC, sensitivity, specificity, and/or C-index of the survival model is closer to 1 than an AUC, sensitivity, specificity, and/or C-index of a survival model where the class-imbalanced data set was not downsampled prior to the survival analysis.

A program storage device readable by a computer, tangibly embodying a program of instructions executable by the computer to perform the method steps for a method for determining disease risk is also disclosed, the method comprising: acquiring a class-imbalanced data set, wherein the class-imbalanced data set comprises biological data from a plurality of subjects, wherein the biological data of each subject includes an observation, a time value, and a plurality of clinical measurements, and wherein the biological data is categorized as being part of a majority data class or a minority data class, wherein the majority data class has a greater number of observations than the minority data class; downsampling the class-imbalanced data set to generate a downsampled data set, wherein the downsampling results in the majority data class having an equivalent or substantially equivalent number of observations as the minority data class; and performing cross-validation on the downsampled data set with a survival analysis to generate a survival model; wherein, the observation comprises an event or no event at a specific time value.

According to aspects of the present disclosure, an AUC, sensitivity, specificity, and/or C-index of the survival model is closer to 1 than an AUC, sensitivity, specificity, and/or C-index of a survival model where the class-imbalanced data set was not downsampled prior to the survival analysis.

A computing system for determining disease risk is also disclosed, the computing system comprising: a memory for storing programmed instructions, and a processor configured to execute the programmed instructions to perform operations comprising: acquiring a class-imbalanced data set, wherein the class-imbalanced data set comprises biological data from a plurality of subjects, wherein the biological data of each subject includes an observation, a time value, and a plurality of clinical measurements, and wherein the biological data is categorized as being part of a majority data class or a minority data class, wherein the majority data class has a greater number of observations than the minority data class; downsampling the class-imbalanced data set to generate a downsampled data set, wherein the downsampling results in the majority data class having an equivalent or substantially equivalent number of observations as the minority data class; and performing cross-validation on the downsampled data set with a survival analysis to generate a survival model; wherein, the observation comprises an event or no event at a specific time value.

According to aspects of the present disclosure, an AUC, sensitivity, specificity, and/or C-index of the survival model is closer to 1 than an AUC, sensitivity, specificity, and/or C-index of a survival model where the class-imbalanced data set was not downsampled prior to the survival analysis.

A non-transitory, computer readable media is also disclosed, wherein the computer readable media has instructions stored thereon that are executable by a processor to perform operations of: acquiring a class-imbalanced data set, wherein the class-imbalanced data set comprises biological data from a plurality of subjects, wherein the biological data of each subject includes an observation, a time value and a plurality of clinical measurements, and wherein the biological data is categorized as being part of a majority data class or a minority data class, wherein the majority data class has a greater number of observations than the minority data class; downsampling the class-imbalanced data set to generate a downsampled data set, wherein the downsampling results in the majority data class having an equivalent or substantially equivalent number of observations as the minority data class; and performing cross-validation on the downsampled data set with a survival analysis to generate a survival model; wherein, the observation comprises an event or no event at a specific time value

According to aspects of the present disclosure, an AUC, sensitivity, specificity, and/or C-index of the survival model is closer to 1 than an AUC, sensitivity, specificity, and/or C-index of a survival model where the class-imbalanced data set was not downsampled prior to the survival analysis.

A computer-implemented method for determining disease risk is also disclosed, the method comprising: receiving with a computer a class-imbalanced data set, wherein the class-imbalanced data set comprises biological data from a plurality of subjects, wherein the biological data of each subject includes an observation, a time value, and a plurality of clinical measurements, and wherein the biological data is categorized as being part of a majority data class or a minority data class, wherein the majority data class has a greater number of observations than the minority data class; downsampling with the computer the class-imbalanced data set to generate a downsampled data set, wherein the downsampling results in the majority data class having an equivalent or substantially equivalent number of observations as the minority data class; and performing with the computer cross-validation on the downsampled data set with a survival analysis to generate a survival model; and wherein the observation comprises an event or no event at a specific time value.

According to aspects of the present disclosure, an AUC, sensitivity, specificity, and/or C-index of the survival model is closer to 1 than an AUC, sensitivity, specificity, and/or C-index of a survival model where the class-imbalanced data set was not downsampled prior to the survival analysis.

### BRIEF DESCRIPTION OF THE DRAWINGS

FIG. 1 illustrates an example of a networked computing environment in which methods, systems, and other aspects of the present disclosure may be implemented.

FIG. 2 is a high-level architecture diagram of a disease risk analysis platform for clinical data acquisition and processing according to the present disclosure.

FIG. 3 illustrates a Kaplan-Meier survival curve for Myocardial Infarction (MI) in the HUNT3 CHD subcohort.

FIG. 4 illustrates Kaplan-Meier survival curves for MI on the test set, stratified by predicted event. For each method, the test set is split into high-risk and average-risk individuals using the threshold identified via cross-validation. Kaplan-Meier curves are then calculated for both groups. In the logistic regression model results, everyone was predicted low risk, thus resulting in only one survival curve.

FIG. 5 illustrates Kaplan-Meier survival curves for MI on the test set, using downsampled Cox elastic net models to predict MI less than or equal to 4 years. Different thresholds for classifying individuals as high-risk were investigated.

### DETAILED DESCRIPTION

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description.

Further, ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise). Any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, “about” or “consisting essentially of” mean±20% of the indicated range, value, or structure, unless otherwise indicated. As used herein, the terms “include” and “comprise” are open ended and are used synonymously.

Although methods and materials similar or equivalent to those described herein can be used in the practice or testing of the present disclosure, suitable methods and materials are described below. All publications, patent applications, patents, and other references mentioned herein are incorporated by reference in their entireties. In case of conflict, the present specification, including explanations of terms, will control. In addition, the materials, methods, and examples are illustrative only and not intended to be limiting.

As used herein, a “SOMAmer” or Slow Off-Rate Modified Aptamer refers to an aptamer having improved off-rate characteristics. SOMAmers can be generated using the improved SELEX methods described in U.S. Pat. No. 7,947,447, entitled “Method for Generating Aptamers with Improved Off-Rates.”

The term “biological sample,” “sample,” and “test sample” are used interchangeably herein to refer to any material, biological fluid, tissue, or cell obtained or otherwise derived from an individual. This includes blood (including whole blood, leukocytes, peripheral blood mononuclear cells, buffy coat, plasma, and serum), sputum, tears, mucus, nasal washes, nasal aspirate, breath, urine, semen, saliva, peritoneal washings, ascites, cystic fluid, meningeal fluid, amniotic fluid, glandular fluid, lymph fluid, nipple aspirate, bronchial aspirate (e.g., bronchoalveolar lavage), bronchial brushing, synovial fluid, joint aspirate, organ secretions, cells, a cellular extract, and cerebrospinal fluid. This also includes experimentally separated fractions of all of the preceding. For example, a blood sample can be fractionated into serum, plasma, or into fractions containing particular types of blood cells, such as red blood cells or white blood cells (leukocytes). In some embodiments, a sample can be a combination of samples from an individual, such as a combination of a tissue and fluid sample. The term “biological sample” also includes materials containing homogenized solid material, such as from a stool sample, a tissue sample, or a tissue biopsy, for example. The term “biological sample” also includes materials derived from a tissue culture or a cell culture. Any suitable methods for obtaining a biological sample can be employed; exemplary methods include, e.g., phlebotomy, swab (e.g., buccal swab), and a fine needle aspirate biopsy procedure. Exemplary tissues susceptible to fine needle aspiration include lymph node, lung, lung washes, BAL (bronchoalveolar lavage), thyroid, breast, pancreas, and liver. Samples can also be collected, e.g., by micro dissection (e.g., laser capture micro dissection (LCM) or laser micro dissection (LMD)), bladder wash, smear (e.g., a PAP smear), or ductal lavage. A “biological sample” obtained or derived from an individual includes any such sample that has been processed in any suitable manner after being obtained from the individual.

As used herein, “biological data” refers to any data derived from a biological sample. Such biological data includes but is not limited to proteomic data which is collected utilizing aptamers specific to protein targets, optionally in a multiplexed aptamer-based assay.

As used herein, “clinical factors” refer to physiological attributes which may be associated with an increased risk of a disease condition or event. Clinical factors include but are not limited to age, weight, blood pressure, height, BMI, cholesterol, and sex.

As used herein, “class-imbalanced” refers to a characteristic of a data set which describes that when the data of the set is classified into two or more classes, the two or more classes have substantially unequal numbers of observations.

As used herein, “cross-validation” refers to any model building and validation technique for assessing model performance on the data used to build the model and how the results of a statistical analysis will generalize to an independent data set, including but not limited to k-fold cross-validation, Monte Carlo cross-validation and leave-p-out validation (wherein p can be from 1 to the total number of samples-1).

As used herein, “downsampling” refers to subsetting of the data of the class with more observations, i.e. the majority data class, to reduce the class-imbalance.

As used herein, “equivalent” or “substantially equivalent” refers to the difference between the compared classes having a less than 10% difference in number of observations.

As used herein, “feature” refers to a measurable property or characteristic for subjects in the data set. Features include but are not limited to protein measurements and clinical factors.

As used herein, “majority data class” refers to the class which has the greater number of observations in a class-imbalanced data set having two classes.

As used herein, “minority data class” refers to the class which has the smaller number of observations in a class-imbalanced data set having two classes.

As used herein, “survival analysis” refers to any modelling of time to event data. Survival analysis methods can be used in any time-to-event outcome, e.g. time to MI, onset of diabetes, onset of various forms of cancer, etc. Survival analysis includes but is not limited to Cox proportional hazard analysis, random forest analysis and accelerated failure time analysis.

As used herein, “survival data set” refers to any data set comprising both time values and event status values that indicate whether the event of interest occurred within the period of time the subject was observed.

In survival analysis, class-imbalance presents a major issue, wherein the number of individuals without a disease (or event) outnumber those with the disease within a certain time frame. This imbalance may result in inaccurate risk predictions for individuals with higher risk of disease. Downsampling mitigates this issue by balancing the number of individuals in the minority and majority class, thus improving the detection and selection of features related to individuals in the minority class as well as their estimated impact on risk of the disease or event occurring.

One context in which downsampling of a class-imbalanced data set for survival analysis has been demonstrated to improve AUC is with proteomics data generated by the SOMAscan® proteomic assay which was used to identify circulating protein biomarkers associated with risk of cardiovascular events in patients with stable Coronary Heart Disease (CHD). The resulting model provides improved ability over existing clinical-risk tools and has broad applicability and generalizability among a composite endpoint of cardiovascular events.

The present disclosure describes a targeted model for predicting secondary MI among patients with stable CHD. Proteomic data were used to identify patients likely to experience secondary MI within four years of a blood draw among patients with stable CHD. In addition to proteomic signals, the data contain information on whether or not specific cardiovascular events occurred over the course of observation, and the length of time to either, a) the event or, b) exiting the study due to other factors. These time-to-event data make the problem well-suited to survival analysis techniques.

When the primary goal is the correct identification of individuals who will have a MI event within 4 years, the analysis can be re-framed as a classification problem, where individuals are the “positive” class if the event occurred before 4 years, and individuals are labeled as the “negative” class if the individual remained in the study beyond the 4-year time-frame with no MI. The use of survival analysis tools improves the predictive accuracy of the model (compared to standard classification models) because survival models “use all the information” by incorporating the time to MI in development of the classifier. This re-framing also allows the use of standard classification metrics, such as the AUC and the confusion matrix to assess model performance. This method of assessing survival models is not a traditional approach, but event-specific classification provides a number of benefits in a clinical setting. Labeling a patient as “positive” or “negative” is more easily understood across a wide audience (compared to, for example, a hazard-ratio or probability). This improved comprehension of a prognostic test allows clinicians to provide more precise, targeted medical management. However, as with standard classification modeling, this approach to survival analysis can suffer from an imbalance in patients who do and do not experience events.

For example, only 8.1% of the individuals in the subcohort analyzed in Example 1 have secondary MI within 4 years, yet more than eight times as many participants (66.9%) survive event-free for longer than four years. The aim of downsampling is to “bias” the classifier so that it pays equal attention to the diagnosed and non-diagnosed individuals to balance the sensitivity and specificity of the model. Re-sampling techniques have been applied to various machine-learning methodologies, but, class-imbalance is an unexplored topic in machine learning using survival modeling techniques.

In Example 1, downsampling is combined with a Cox proportional hazards elastic net regression model, and prediction of an MI event within 4 years of initial blood draw is assessed.

As is apparent from Example 1, the performance of a survival analysis, e.g. a-Cox proportional hazards elastic net model (i.e., a “Coxnet” model), can be improved by downsampling the data during modeling. The present disclosure effectively demonstrates that a downsampled Coxnet model was superior to a standard Coxnet model, a downsampled elastic net logistic regression model, and a standard elastic net logistic regression model.

In addition to downsampling, there are other methods for handling class-imbalance that could also be incorporated into survival models. For example, case-weighting, simple oversampling, or more complex oversampling techniques such as the Synthetic Minority Oversampling Technique (SMOTE) can be considered with traditional survival analysis, as well as expanded machine learning methods such as random survival forests.

While the Example 1 describes in detail the combination of downsampling in survival analysis in the context of prediction of an MI event within the specified time-frame, the methods disclosed herein can be applied to any prediction of a disease condition or a disease-related event risk within a selected time-frame.

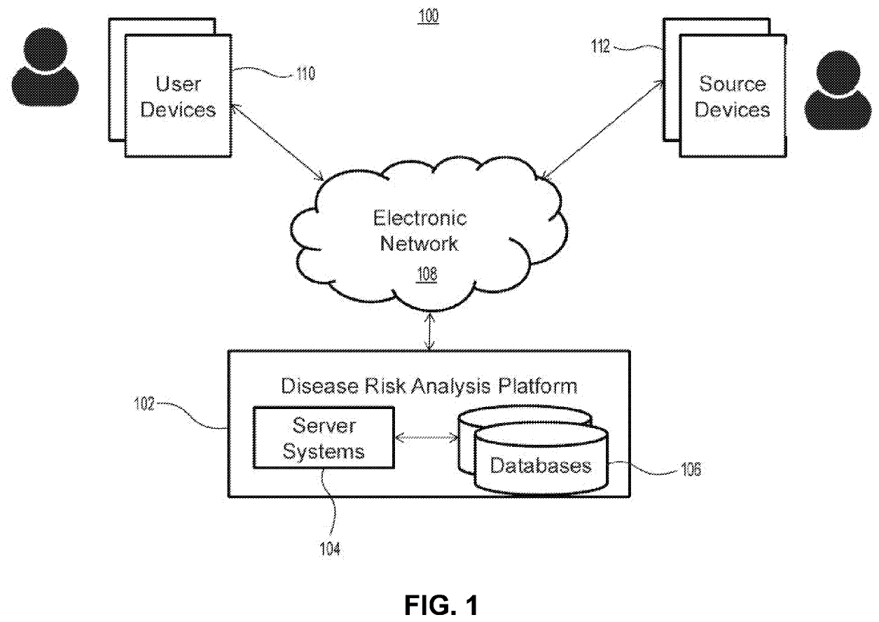

FIG. 1 is a block diagram of a networked computing environment 100 for processing electronic data to determine disease risk, for example by downsampling class-imbalanced data, according to aspects of the disclosure. As shown in FIG. 1, the networked computing environment 100 may include a disease risk analysis platform 102, including server systems 104 and electronic databases 106. The server systems 104 may store and execute software modules, algorithms, or other subsystems of the disease risk analysis platform 102 for use through an electronic network 108, such as the Internet. Users may access the disease risk analysis platform 102 through the electronic network 108 by user devices 110, such as a computing device or the like. User devices 110 may allow a user to display a web browser for accessing the disease risk analysis platform 102 hosted by the server system 104 through the electronic network 108. The user devices 110 may be any type of device for accessing web pages, such as personal computing devices, mobile computing devices, or the like. Source devices 112 may provide and/or receive data to/from the disease risk analysis platform 102 through the electronic network 108. The source devices 112 may be any type of device for accessing web pages, such as personal computing devices, mobile computing devices, or the like.

FIG. 1 is provided merely as an example. Other examples are possible and may differ from networked computing environment 100 of FIG. 1. In addition, the number and arrangement of devices and networks shown in networked computing environment 100 are provided as an example. In practice, there may be additional devices, fewer devices and/or networks, different devices and/or networks, or differently arranged devices and/or networks than those shown in networked computing environment 100. Furthermore, two or more devices shown in FIG. 1 may be implemented within a single device, or a single device shown in FIG. 1 may be implemented as multiple, distributed devices. Additionally, or alternatively, one or more user devices and/or server systems of networked computing environment 100 may perform one or more functions of the server system 104 and/or the disease risk analysis platform 102.

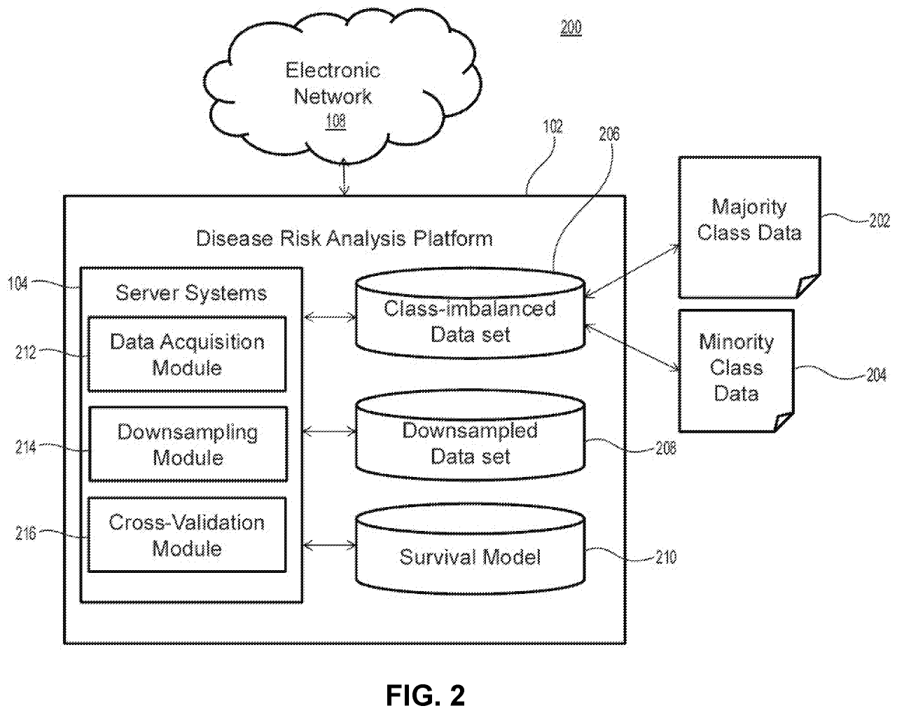

FIG. 2 depicts an exemplary computer architecture 200 for processing electronic data to determine disease risk. Specifically, FIG. 2 depicts an exemplary computer architecture 200 configured for combining downsampling of class-imbalanced sets with survival analysis, according to one or more embodiments of the present disclosure. As shown in the computer architecture 200 of FIG. 2, server systems 104 of disease risk analysis platform 102 may comprise a data acquisition module 212, a downsampling module 214, and a cross-validation module 216. Disease risk analysis platform 102 may further comprise one or more databases or data stores, whether locally or remotely accessed. For example, as shown in FIG. 2, disease risk analysis platform 102 may comprise a class-imbalanced data set 206 comprising majority class data 202 and minority class data 204. Disease risk analysis platform 102 may further comprise a downsampled data set 208 and a survival model 210. It should be appreciated that one or more of data acquisition module 212, downsampling module 214, cross-validation module 216, class-imbalanced data set 206, downsampled data set 208, and survival model 210 may have some or all of its functions and contents stored or executed locally, remotely, or both locally and remotely, and that functions thereof may be combined or distributed across other components of the platform.

In one embodiment of the exemplary computer architecture 200, the data acquisition module 212 may receive, from user devices 110 or source devices 112, class-imbalanced data set 206, comprising majority class data 202 and minority class data 204. This class-imbalanced data set 206 may be processed by the downsampling module 214, to produce the downsampled data set 208. This downsampled data set 208 may be processed by the cross-validation module 216 to produce the survival model 210. This survival model 210 may be then be sent via the electronic network 108 to user devices 100 and/or source devices 112.

If programmable logic is used, such logic may execute on a commercially available processing platform or a special purpose device. One of ordinary skill in the art may appreciate that embodiments of the disclosed subject matter can be practiced with various computer system configurations, including multi-core multiprocessor systems, minicomputers, mainframe computers, computer linked or clustered with distributed functions, as well as pervasive or miniature computers that may be embedded into virtually any device.

For instance, at least one processor device and a memory may be used to implement the above-described embodiments. A processor device may be a single processor, a plurality of processors, or combinations thereof. Processor devices may have one or more processor “cores.”

Various embodiments of the present disclosure, as described above in the examples of FIGS. 1 and 2 may be implemented using a processor device. After reading this description, it will become apparent to a person skilled in the relevant art how to implement embodiments of the present disclosure using other computer systems and/or computer architectures. Although operations may be described as a sequential process, some of the operations may in fact be performed in parallel, concurrently, and/or in a distributed environment, and with program code stored locally or remotely for access by single or multi-processor machines. In addition, in some embodiments the order of operations may be rearranged without departing from the spirit of the disclosed subject matter.

It should be appreciated that the disease risk analysis platform 102 and/or any device used for accessing the disease risk analysis platform 102, such as user device 110 or source device 112, may include a central processing unit (CPU). Such a CPU may be any type of processor device including, for example, any type of special purpose or a general-purpose microprocessor device. As will be appreciated by persons skilled in the relevant art, a CPU also may be a single processor in a multi-core/multiprocessor system, such system operating alone, or in a cluster of computing devices operating in a cluster or server farm. A CPU may be connected to a data communication infrastructure, for example, a bus, message queue, network, or multi-core message-passing scheme.

It should further be appreciated that the disease risk analysis platform 102 and/or any device used for accessing the disease risk analysis platform 102, such as user device 110 or source device 112, may also include a main memory, for example, random access memory (RAM), and may also include a secondary memory. Secondary memory, e.g., a read-only memory (ROM), may be, for example, a hard disk drive or a removable storage drive. Such a removable storage drive may comprise, for example, a floppy disk drive, a magnetic tape drive, an optical disk drive, a flash memory, or the like. The removable storage drive in this example reads from and/or writes to a removable storage unit in a well-known manner. The removable storage unit may comprise a floppy disk, magnetic tape, optical disk, etc., which is read by and written to by the removable storage drive. As will be appreciated by persons skilled in the relevant art, such a removable storage unit generally includes a computer usable storage medium having stored therein computer software and/or data.

In alternative implementations, secondary memory may include other similar means for allowing computer programs or other instructions to be loaded into a device. Examples of such means may include a program cartridge and cartridge interface (such as that found in video game devices), a removable memory chip (such as an EPROM, or PROM) and associated socket, and other removable storage units and interfaces, which allow software and data to be transferred from a removable storage unit to device.

It should further be appreciated that the disease risk analysis platform 102 and/or any device used for accessing the disease risk analysis platform 102, such as user device 110 or source device 112, may also include a communications interface (“COM”). Communications interface allows software and data to be transferred between device and external devices. Communications interface may include a modem, a network interface (such as an Ethernet card), a communications port, a PCMCIA slot and card, or the like. Software and data transferred via communications interface may be in the form of signals, which may be electronic, electromagnetic, optical, or other signals capable of being received by communications interface. These signals may be provided to communications interface via a communications path of device, which may be implemented using, for example, wire or cable, fiber optics, a phone line, a cellular phone link, an RF link, or other communications channels.

The hardware elements, operating systems and programming languages of such equipment are conventional in nature, and it is presumed that those skilled in the art are adequately familiar therewith. A device used for accessing the disease risk analysis platform also may include input and output ports to connect with input and output devices such as keyboards, mice, touchscreens, monitors, displays, etc. Of course, the various server functions may be implemented in a distributed fashion on a number of similar platforms, to distribute the processing load. Alternatively, the servers may be implemented by appropriate programming of one computer hardware platform.

The systems, apparatuses, devices, and methods disclosed herein are described in detail by way of examples and with reference to the figures. The examples discussed herein are examples only and are provided to assist in the explanation of the apparatuses, devices, systems, and methods described herein. None of the features or components shown in the drawings or discussed below should be taken as mandatory for any specific implementation of any of the apparatuses, devices, systems, or methods unless specifically designated as mandatory. For ease of reading and clarity, certain components, modules, or methods may be described solely in connection with a specific figure. In this disclosure, any identification of specific techniques, arrangements, etc. are either related to a specific example presented or are merely a general description of such a technique, arrangement, etc. Identifications of specific details or examples are not intended to be, and should not be, construed as mandatory or limiting unless specifically designated as such. Any failure to specifically describe a combination or sub-combination of components should not be understood as an indication that any combination or sub-combination is not possible. It will be appreciated that modifications to disclosed and described examples, arrangements, configurations, components, elements, apparatuses, devices, systems, methods, etc. can be made and may be desired for a specific application. Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

Throughout this disclosure, references to components or modules generally refer to items that logically can be grouped together to perform a function or group of related functions. Components and modules can be implemented in software, hardware, or a combination of software and hardware. The term “software” is used expansively to include not only executable code, for example machine-executable or machine-interpretable instructions, but also data structures, data stores and computing instructions stored in any suitable electronic format, including firmware, and embedded software. The terms “information” and “data” are used expansively and includes a wide variety of electronic information, including executable code; content such as text, video data, and audio data, among others; and various codes or flags. The terms “information,” “data,” and “content” are sometimes used interchangeably when permitted by context.

### EXAMPLES

The following examples are presented in order to more fully illustrate some embodiments of the invention. They should in no way be construed, however, as limiting the broad scope of the invention. Those of ordinary skill in the art can readily adopt the underlying principles of this discovery to design various compounds without departing from the spirit of the current invention.

### Example 1

This example provides a description of downsampling combined with a Cox proportional hazards elastic net regression model to assess prediction of a myocardial infarction (MI) event within 4 years of initial blood draw, as can be done within the exemplary data risk analysis platform of FIG. 2.

The purposes of this example were at least two-fold: 1) selection and identification of features that predict both the minority and majority classes, and 2) derivation of estimated effect sizes such that the risk for the minority class is well-predicted. For contrast, the predictive capabilities of logistic regression elastic net models were examined (with and without downsampling) as well as a Cox elastic net model without downsampling.

Materials and Methods—Dataset

Samples used in analysis were a subcohort from the HUNT3 study, a prospective cohort study from Norway which included blood samples drawn from study participants and follow-up health information. The CHD subcohort was described previously (Peter Ganz, et al. Development and validation of a protein-based risk score for cardiovascular outcomes among patients with stable coronary heart disease. Jama, 315(23):2532-2541, 2016), with inclusion criteria directed for evidence of existing but stable CHD via a history of MI more than six months previous, stenosis, inducible ischemia, or previous coronary revascularization. Plasma samples were assayed using the SOMAscan® Assay (SomaLogic, Inc; Boulder, Colo. USA), which uses Slow Off-rate Modified Aptamer (SOMAmer®) reagents to measure relative protein abundance. The V4 assay measures 5,220 protein analytes and is a well-established platform for protein biomarker discovery.

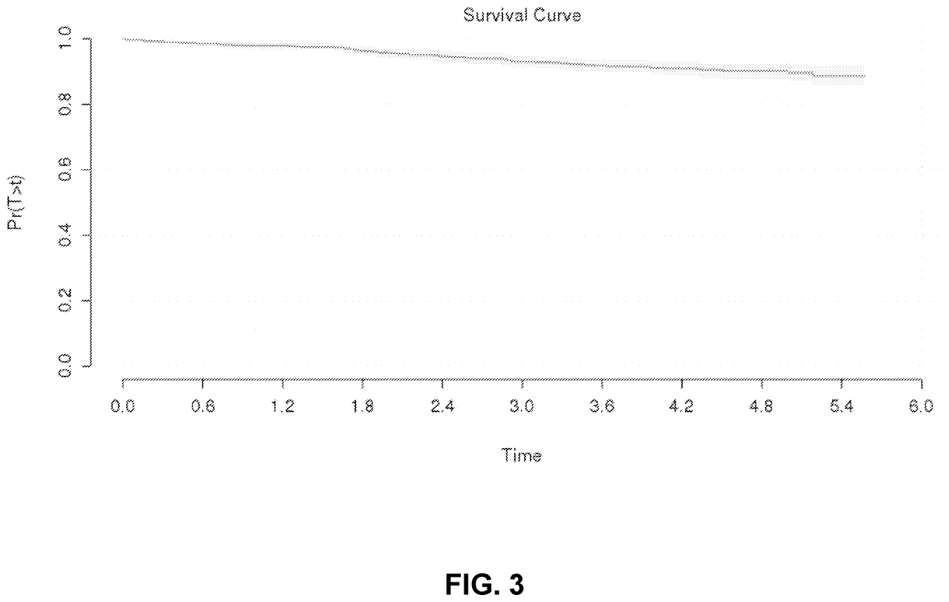

In the subcohort, 8.1% of patients experienced a secondary MI within 4 years (Table 1). The Kaplan-Meier survival curve for MI in the CHD subcohort is depicted in FIG. 3. The Kaplan-Meier curve is an empirical, non-parametric method for examining how the probability of being event-free (e.g., MI-free) changes over time. There is a gradual decrease in the probability of being event-free for MI in the CHD subcohort of the HUNT3 dataset. Table 1 shows incidence of MI and demographic information in the CHD subcohort.

TABLE 1
Demographic Characteristics for Stable CHD Subcohort

| | MI within | | Non-MI within | | No event (MI or | | | |
|---|---|---|---|---|---|---|---|---|
| Characteristic | 4 years | | 4 years | | other) >4 years | | Total | |
| Number of subjects | 61 | (8.1%) | 189 | (25.0%) | 506 | (66.9%) | 756 | (100%) |
| (% total) | | | | | | | | |
| Gender, Female | n = 20 | (32.8%) | n = 57 | (30.2%) | n = 128 | (25.3%) | n = 205 | (27.1%) |
| (% of group) | | | | | | | | |
| Gender, Male | n = 41 | (67.2%) | n = 132 | (69.8%) | n = 378 | (74.7%) | n = 551 | (72.9%) |
| (% of group) | | | | | | | | |
| Mean Age, years | 72.6 ± 11.1 | | 72.8 ± 10.3 | | 67.7 ± 10.2 | | 69.4 ± 10.5 | |
| (±SD) | | | | | | | | |
| Time to event, years | 1.93 | (1.97) | 2.57 | (2.73) | 4.69 | (0.75) | 4.37 | (1.06) |
| (IQR) | | | | | | | | |

Materials and Methods—Cox Elastic Net Models

Survival data is characterized by an outcome that is the time to an event, which accommodates a wide range of topics, including an MI event, death from cancer, re-hospitalization for a disease, a machine component failing, and more. The nature of time-dependent data is that the event will not be observed for some individuals if it occurs outside the study period. These individuals are “censored,” which can occur for multiple reasons (e.g., death from non-MI related causes, individuals withdrawing from the study, MI occurring after the study window ending). While there are multiple types of censoring, the data contains right-censored individuals, meaning that for patients who do not have an MI event, it is assumed to have occurred after the last observed time point.

Survival data is characterized through a survival function, S(.), which is the probability of being event-free and is calculated at time point t as

S(t)=P(T>t)=∫ₜ^{∞}f(d)du,

where f(.) is the probability density function of time to MI. Along with survival function, features that significantly increase or decrease time-to-event may also be identified and characterized. While there are numerous survival analysis techniques, one of the most common is the Cox proportional hazards model. The Cox model is expressed as

λ(t|Xᵢ,β)=λ₀(t)exp{X′ᵢβ}.

Here, λ(t|.) is the hazard function (or “immediate risk of failure” function) and is defined as λ(t|.)=f(t|.)/S(t|.). Additionally, Xᵢ is a p×1 vector of feature measurements for the i^{th} individual, and β is a p×1 vector of feature effects. The primary goal of the Cox model is to estimate the effects features have on an individual's risk of the event occurring. The baseline hazard rate, λ₀(t), is treated as a nuisance parameter in the estimation routine and therefore is not examined.

Because the number of features in the data set is greater than the sample size, an elastic net penalty may be incorporated into our model, a form of penalized regression that combines the least absolute shrinkage and selection operator (i.e. lasso) and ridge regression or Tikhonov regularization. This tool performs feature selection through the lasso routine while allowing correlated features to remain in the model together, such that p can be greater than n. In a standard regression model, the feature effects, β, are typically estimated by minimizing the difference between the response, Yᵢ, and the predictors, X′ᵢβ. However, with elastic net regularization, the estimated feature effects are calculated as

$$\hat{β} = {argmin}_{β} {\vert Y - X β \vert}^{2} + {λ}_{2} {\vert β \vert}^{2} + {λ}_{1} \vert β \vert ,$$

where λ₁ is a L₁ penalty associated with lasso regression, and λ₂ is a L₂ penalty associated with ridge regression.

Survival analysis was combined with the elastic net penalty by using the Cox elastic net model implemented via the glmnet package available in CRAN-R. The Cox elastic net model merges the standard Cox proportional hazards model with elastic net penalization, allowing use of survival techniques to develop a classifier, plus the benefits of penalized regression.

To mitigate class-imbalance, Cox proportional hazard elastic net models were combined with downsampling techniques. This approach allowed identification of features that best predict whether an individual is at “high-risk” of having a MI event within 4 years, with the “high-risk” classifier calculated using hazard ratio threshold that is identified via cross-validation. Additionally, this technique estimated the feature effects in a way that allows features that accurately predict high-risk individuals to have different “weights” (i.e., β estimates) than they would if derived using the full cohort.

For comparison, two elastic net logistic regression models (with or without downsampling, may be implemented via the caret package in R), as well as a Cox elastic net model that did not incorporate downsampling techniques. Models were compared using AUC, sensitivity, specificity and C-Index, as appropriate.

Analyses were performed using R version 3.4.4 in RStudio server version 1.1.453.

Materials and Methods—Data Subsetting

The dataset was split into a training set (80% of the data) and a test set (20%). The training set was used for model building and the final models were evaluated on the test set. Thresholds for prediction on the test set for Cox elastic net models were calculated as the average of the thresholds generated per fold during cross-validation. Before implementing the penalized regression models, univariate filtering was performed using the training set. Student's t-tests were calculated per analyte to assess if mean values were statistically significantly different between individuals who did and did not have an MI event in the study window. For consistency in demonstrating utility of the technique, the top 100 analytes (ranked by false discovery rate values) were included across model development.

Results

Results of the downsampled Cox elastic net model were compared with two logistic regression elastic net models (downsampled and not) and a Cox elastic net model that did not use downsampling. For simplicity of notation, the Cox elastic net models are referred to as “Coxnet” models and the elastic net logistic regression models as “LRnet” models. For models that are downsampled, “DS” was prepended (e.g., the Cox elastic net model that implements downsampling is “DS-Coxnet”).

Across models, five repeats of 5-fold cross-validation were used on the training set to select the optimal models within each model type. Optimal models were selected via maximum AUC. Feature selection, the estimated effects, and the classification threshold were allowed to differ across models. Following cross-validation, the predictive capabilities of the top model in each category were evaluated on the test data set.

During model development, the Coxnet models were created using the original data but were optimized for classification using the AUC metric at the 4-year time point. This means that a standard survival model was built, but a binary 4 year-mark classifier (yes/no MI before four years) was used to calculate AUC and optimize the model. The 4-year outcome was used in development of the logistic regression models, which were also optimized using AUC. The C-Index was calculated for the survival models for the purpose of model comparison using a standard survival model metric.

Model Results and Comparison

Cross-validation results show that both Coxnet models vastly outperform the standard LRnet model (see Table 2). This result is expected, as survival analysis methods use the time to the event information as part of feature selection and model development. A more compelling result is that the DS-Coxnet model outperformed both the DS-LRnet and standard Coxnet models across all classification metrics (AUC, sensitivity, specificity). Additionally, the DS-Coxnet model has a higher C-Index than the standard Coxnet model, indicating that the downsampled model better predicts the ordering of times to MI.

TABLE 2
Cross-validated training set results

| Model | Tuning parameters | AUC | Sensitivity | Specificity | C-Index |
|---|---|---|---|---|---|
| Downsampled Cox model | α = 0.75, λ = 0.1 | 0.78 | 0.74 | 0.79 | 0.74 |
| Cox model | α = 0.75, λ = 0.05 | 0.61 | 0.67 | 0.66 | 0.58 |
| Downsampled Logistic Regression | α = 0.75, λ = 0.05 | 0.75 | 0.65 | 0.73 | — |
| Logistic Regression | α = 0.75, λ = 0.05 | 0.58 | 0 | 1 | — |

Following model optimization via cross-validation, the predictive abilities of the top models were evaluated on the test set, including an examination of sensitivity and specificity based on correctly predicting an individual as “high-risk” of having an MI by the 4-year mark. Performance metrics for all the models on the test set are shown in Table 3. The DS-Coxnet model is the only model that performs better than “random chance” with an AUC of 0.63. Furthermore, the DS-Coxnet model has the highest sensitivity and specificity compared to both the DS-LRnet model and the standard Coxnet model (unsurprisingly, the LRnet model performs as poorly on the test data set as it did on the training data set).

TABLE 3
Test set results

| Model | Threshold | AUC | Sensitivity | Specificity | C-Index |
|---|---|---|---|---|---|
| Downsampled Cox model | 0.46 | 0.63 | 0.46 | 0.80 | 0.74 |
| Cox model | −0.004 | 0.49 | 0.38 | 0.56 | 0.49 |
| Downsampled Logistic Regression | 0.50 | 0.54 | 0.15 | 0.72 | — |
| Logistic Regression | 0.50 | 0.49 | 0 | 1 | — |

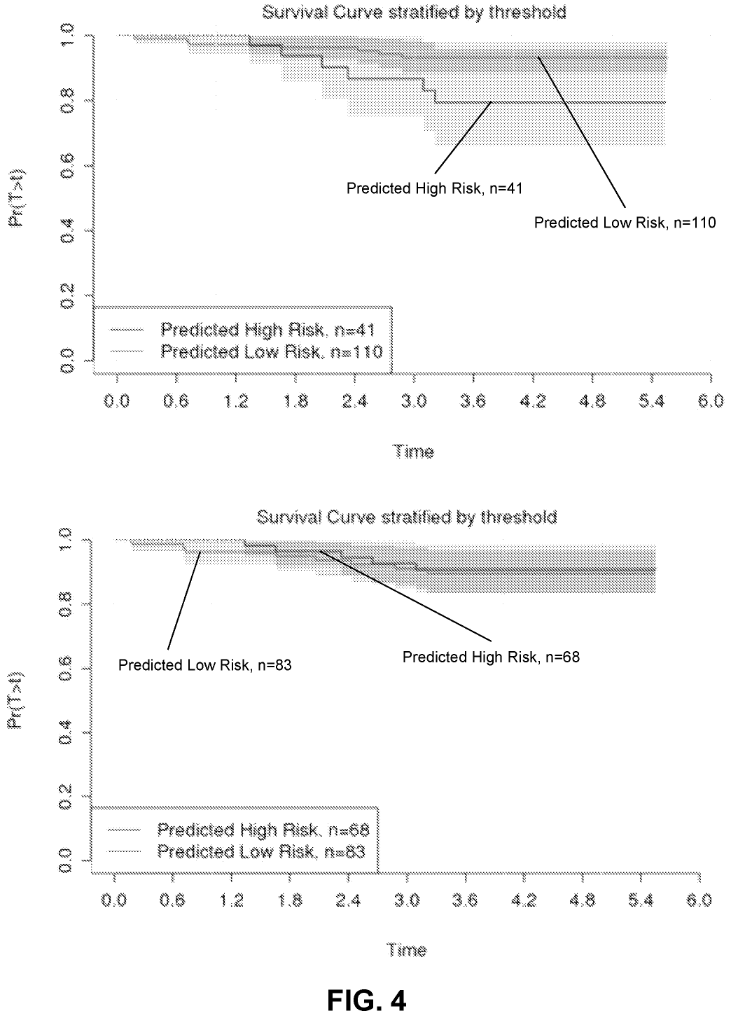

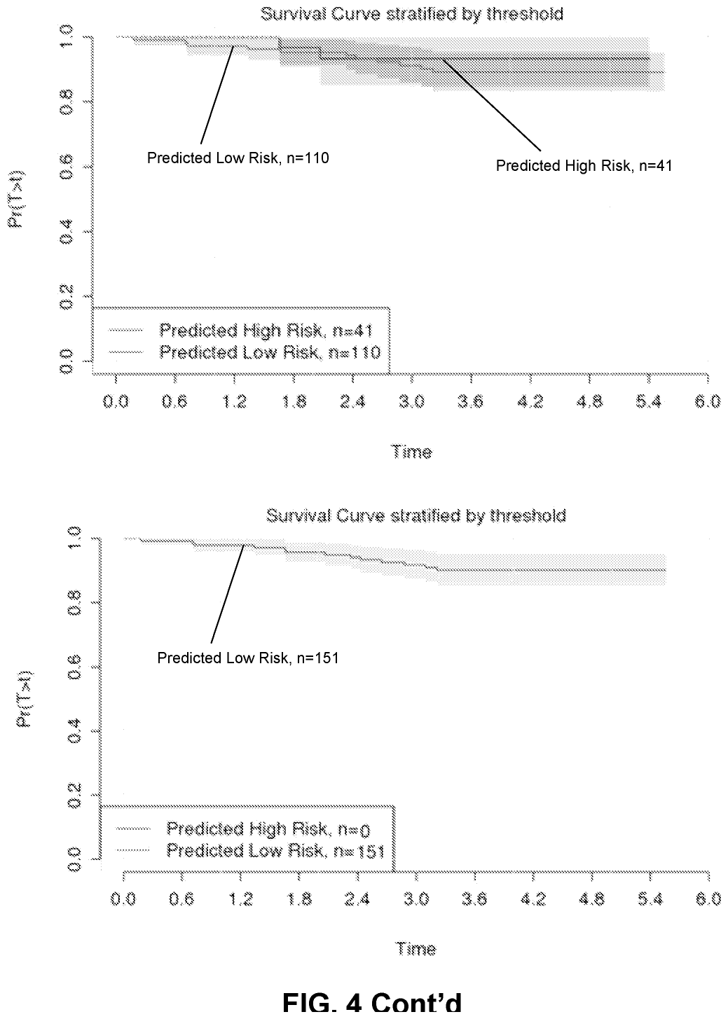

To further demonstrate the benefit of the downsampled survival model approach, for each model, Kaplan-Meier curves were generated on the test set, stratified by whether an individual is predicted as high-risk or not using the model-specific threshold values identified through cross-validation (see FIG. 4). For this comparison, thresholds for the standard and DS-Coxnet model were calculated as the mean threshold values across the cross-validation iterations. This method of visual inspection shows a very clear separation between the high-risk and average-risk groups using the threshold for the DS-Coxnet model. This separation is not as well-defined for the other models.

The combined evidence of the figures and model assessment metrics (Table 3) make a compelling case that the downsampled survival model approach is beneficial in identifying individuals at high-risk of MI within four years.

Threshold Investigation for Downsampled Coxnet Model

The threshold used for predicting the test set using the DS-Coxnet model was the mean across all the thresholds from the cross-validation iterations. While this threshold resulted in a higher sensitivity and specificity than other models, those values were still quite imbalanced. An important consideration is whether the sensitivity/specificity trade-off can be further balanced by manipulating the threshold for prediction.

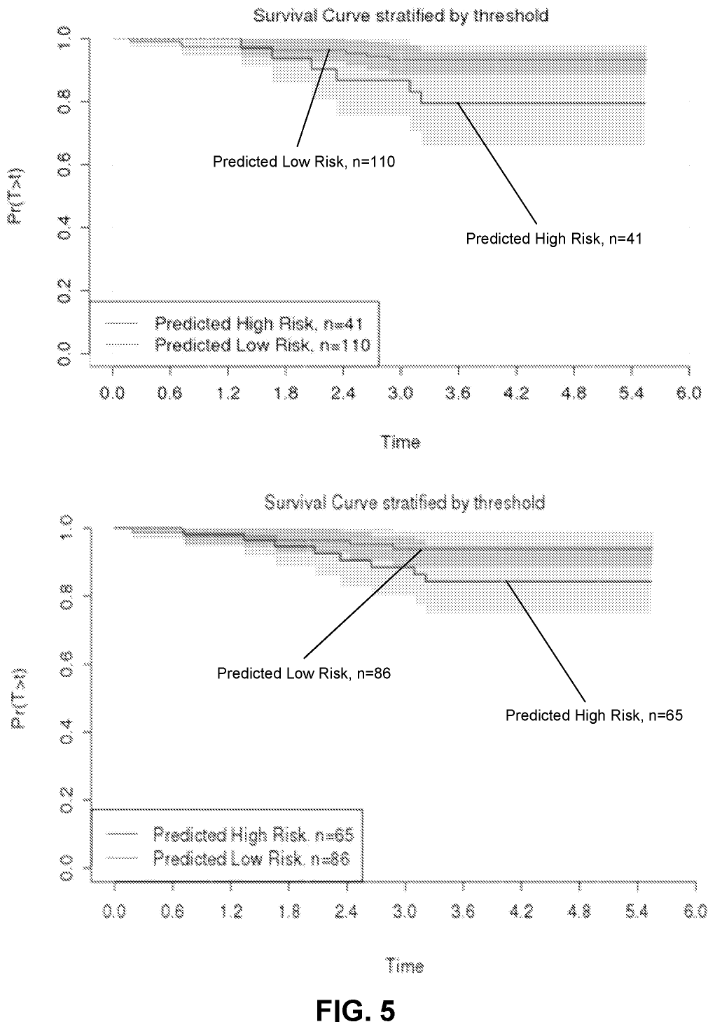

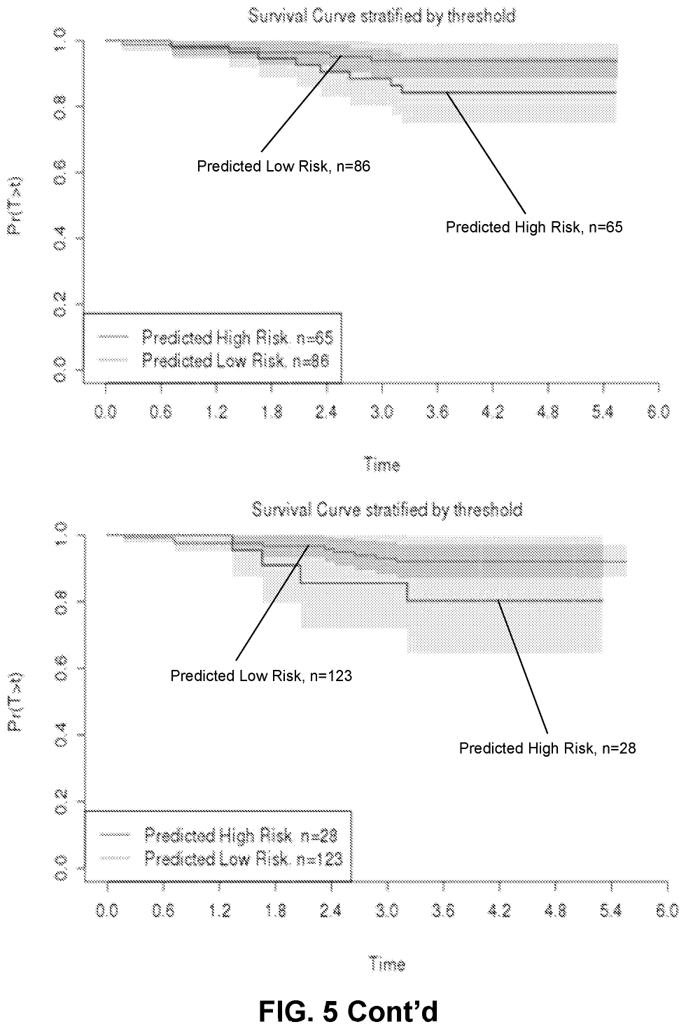

As with classification models, the threshold can be adjusted to find values that maximize sensitivity, maximize specificity, or minimize the difference between sensitivity and specificity on the test set. Table 4 displays the performance metrics of the different thresholds on the test set, and FIG. 5 plots the Kaplan-Meier curves for each. As shown in Table 4, varying the thresholds for predictions results in sensitivities higher than 60% without a decrease in AUC. However, the Kaplan-Meier curves (FIG. 5) show the widest separation between high-risk and average-risk individuals using the mean threshold value.

TABLE 4
Applying different thresholds on the test
set using a downsampled Cox model

| Experiment | Threshold | AUC | Sensitivity | Specificity |
|---|---|---|---|---|
| Mean threshold | 0.46 | 0.63 | 0.46 | 0.80 |
| Balanced sens & spec | 0.165 | 0.63 | 0.62 | 0.64 |
| Maximize sensitivity | 0.165 | 0.63 | 0.62 | 0.64 |
| Maximize specificity | 0.712 | 0.58 | 0.31 | 0.85 |

While the sensitivity and specificity remain relatively lower than typically desired (i.e., 70% or more), this result is likely due to the fact that there are only 13 subjects in the test set who had an MI event before four years, limiting model development. However, the analysis demonstrates that the threshold used for classifying risk levels in survival models can be adjusted in the same way as in classification models.

It is intended that the specification and examples be considered as exemplary only, with a true scope and spirit of the disclosure being indicated by the following claims.

1. A method comprising:
a) acquiring a class-imbalanced data set, wherein the class-imbalanced data set comprises biological data from a plurality of subjects, wherein the biological data of each subject includes an observation, a time value, and a plurality of clinical measurements, and wherein the biological data is categorized as being part of a majority data class or a minority data class, wherein the majority data class has a greater number of observations than the minority data class;
b) downsampling the class-imbalanced data set to generate a downsampled data set, wherein the downsampling results in the majority data class having an equivalent or substantially equivalent number of observations as the minority data class; and
c) performing cross-validation on the downsampled data set with a survival analysis to generate a survival model;
wherein the observation comprises an event or no event at a specific time value, wherein an AUC, sensitivity, specificity, and/or C-index of the survival model is closer to 1 than a AUC, sensitivity, specificity, and/or C-index of a survival model where the class-imbalanced data set was not downsampled prior to the survival analysis.

2. (canceled)

3. The method of claim 1, wherein the class-imbalanced data set is a survival data set.

4. The method of claim 1, wherein the event is a disease, disorder, or condition of a subject.

5. The method of claim 1, wherein the survival analysis is selected from the group consisting of a Cox proportional hazard analysis, a random forest analysis, an accelerated failure time analysis, and any combination thereof.

6. The method of claim 5, further comprising an elastic net penalty.

7. The method of claim 1, wherein the cross-validation is at least a 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold cross-validation.

8. The method of claim 1, wherein the survival model comprises from 5 to 1,000 features, wherein each feature is selected from the group consisting of a protein measurement, a clinical factor, and a combination thereof.

9. The method of claim 8, wherein the clinical factor is selected from the group consisting of age, weight, blood pressure, height, BMI, cholesterol, sex, and a combination thereof.

10. The method of claim 1, wherein the clinical measurements are selected from proteomic measurements, genomic measurements, transcriptome measurements, metabolomics measurements, or a combination thereof.

11. The method of claim 1, wherein the cross-validation is selected from k-fold cross-validation, Monte Carlo cross-validation, and Leave N Out validation.

12. The method of claim 1, wherein the majority data class is 95% of the class-imbalanced data set and the minority data class is 5% of the class-imbalance data set.

13. The method of claim 1, wherein the majority data class is 90% of the class-imbalanced data set and the minority data class is 10% of the class-imbalance data set.

14. The method of claim 1, wherein the majority data class is 85% of the class-imbalanced data set and the minority data class is 15% of the class-imbalance data set.

15. The method of claim 1, wherein the majority data class is 80% of the class-imbalanced data set and the minority data class is 20% of the class-imbalance data set.

16. The method of claim 1, wherein the majority data class is 75% of the class-imbalanced data set and the minority data class is 25% of the class-imbalance data set.

17. The method of claim 1, wherein the majority data class is 70% of the class-imbalanced data set and the minority data class is 30% of the class-imbalance data set.

18. The method of claim 1, wherein the majority data class is 65% of the class-imbalanced data set and the minority data class is 35% of the class-imbalance data set.

19. The method of claim 1, wherein the majority data class is 60% of the class-imbalanced data set and the minority data class is 40% of the class-imbalance data set.

20-32. (canceled)
